(19) United States
(12) Patent Application Publication
(10) Pub. No.: US 2008/0233043 A1
(43) Pub. Date: Sep. 25, 2008

(54) Method For the Production of Chlorine By Means of Gas Phase Oxidation of Hydrogen Chloride

(75) Inventors: Gerhard Olbert, Dossenheim (DE); Olga Schubert, Ludwigshafen (DE); Martin Sesing, Waldsee (DE); Eckhard Stroefer, Mannheim (DE); Martin Fiene, Niederkirchen (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Akiengesellschaft, Ludwigshafen (DE)

(21) Appl. No.: 10/584,055

(22) PCT Filed: Dec. 23, 2004
(86) PCT No.: PCT/EP2004/014671

§ 371 Date: Jun. 22, 2006

(30) Foreign Application Priority Data
Dec. 23, 2003 (DE) 103 61 519.9

(51) Int. Cl. C01B 7/04; C07C 17/08

(52) U.S. Cl. 423/502; 570/248

(57) ABSTRACT

A process for preparing chlorine by gas-phase oxidation of hydrogen chloride by means of a gas stream comprising molecular oxygen in the presence of a fixed-bed catalyst, wherein the process is carried out in a reactor (1) having heat-exchange plates (2) which are arranged in the longitudinal direction of the reactor (1) and have a spacing between them and through which a heat transfer medium flows, inlet and outlet facilities (3, 4) for the heat transfer medium to the heat-exchange plates (2) and also gaps (5) between the heat-exchange plates (2) in which the fixed-bed catalyst is present and into which the hydrogen chloride and the gas stream comprising molecular oxygen are passed, is described.

The invention relates to a process for preparing chlorine by gas-phase oxidation of hydrogen chloride in the presence of a fixed-bed catalyst.

The process developed by Deacon in 1868 for the catalytic oxidation of hydrogen chloride by means of oxygen in an exothermically equilibrium reaction is the start of industrial chlorine chemistry. Chloralkali electrolysis has pushed the Deacon process far into the background, and virtually all chlorine produced is obtained by electrolysis of aqueous sodium chloride solutions.

However, the attractiveness of the Deacon process has recently been increasing again, since the worldwide demand for chlorine is growing more strongly than the demand for sodium hydroxide. This development makes the process for preparing chlorine by oxidation of hydrogen chloride, which is decoupled from sodium hydroxide production, attractive. In addition, hydrogen chloride is obtained in large amounts as coproduct in, for example, phosgenation reactions, for instance in isocyanate production. The hydrogen chloride formed in isocyanate production is predominantly used in the oxychlorination of ethylene to 1,2-dichloroethane which is processed to give vinyl chloride and further to PVC. Examples of further processes in which hydrogen chloride is obtained are the preparation of vinyl chloride, polycarbonate production and the recycling of PVC.

The oxidation of hydrogen chloride to chlorine is an equilibrium reaction. The position of the equilibrium shifts away from the desired end product as the temperature increases. It is therefore advantageous to use catalysts which have a very high activity and allow the reaction to proceed at relatively low temperatures. Such catalysts are in particular, catalysts based on copper or catalysts based on ruthenium, for example the supported catalysts described in DE-A 197 48 299 comprising the active composition ruthenium oxide or ruthenium mixed oxide, with the ruthenium oxide content being from 0.1 to 20% by weight and the mean particle diameter of ruthenium oxide being from 1.0 to 10.0 nm. Further supported catalysts based on ruthenium are known from DE-A 197 34 412: ruthenium chloride catalysts comprising at least one of the compounds titanium oxide and zirconium oxide, ruthenium-carbonyl complexes, ruthenium salts of inorganic acids, ruthenium-nitrosyl complexes, ruthenium-amine complexes, ruthenium complexes of organic amines or ruthenium-acetylacetonate complexes. In addition to ruthenium, gold can also be present in the active composition of the catalyst.

A known industrial problem in gas-phase oxidations, here the oxidation of hydrogen chloride to chlorine, is the formation of hot spots, i.e. places of local overheating, which can lead to destruction of the catalyst material and catalyst tube material. To reduce or prevent the formation of hot spots, it has therefore been proposed in WO 01/60743 that catalyst charges which have different activities in different regions of the catalyst tubes, i.e. catalysts having an activity matched to the temperature profile of the reaction, be used. A similar result is said to be achieved by targeted dilution of the catalyst bed with inert material.

In the hot spot regions, in particular at temperatures above 400° C., the ruthenium-containing catalyst is damaged, in particular by formation of volatile ruthenium oxide.

Against this background, it was an object of the invention to provide a process for preparing chlorine on an industrial scale by gas-phase oxidation of hydrogen chloride by means of a gas stream comprising molecular oxygen in the presence of a fixed-bed reactor, which process ensures effective removal of heat and has a satisfactory period of operation despite the highly corrosive reaction mixture. Furthermore, the process should reduce or avoid the hot spot problem without a decrease in the catalyst activity, or with a relatively small decrease in the catalyst activity, and without dilution of the catalyst, and also reduce or avoid catalyst damage as a result of hot spot formation.

In one embodiment, it was an object of the invention to provide a process for starting up and shutting down the reactor for preparing chlorine by gas-phase oxidation of hydrogen chloride, which process reduces the corrosion problems.

We have accordingly found a process for preparing chlorine by gas-phase oxidation of hydrogen chloride by means of a gas stream comprising molecular oxygen in the presence of a fixed-bed catalyst, wherein the process is carried out in a reactor having heat-exchange plates which are arranged in the longitudinal direction of the reactor and have a spacing between them and through which a heat transfer medium flows, inlet and outlet facilities for the heat transfer medium to the heat-exchange plates and also gaps between heat-exchange plates in which the fixed-bed catalyst is present and into which the hydrogen chloride and the gas stream comprising molecular oxygen are passed.

In the Deacon process, the reaction temperatures are usually in the range from 150 to 500° C. and the reaction pressure is from 1 to 25 bar.

Since the reaction is an equilibrium reaction, it is advantageous to work at the lowest possible temperatures at which the catalyst still has a satisfactory activity. Furthermore, it is advantageous to use oxygen in superstoichiometric amounts. For example, a two- to four-fold oxygen excess is customary. Since no decreases in selectivity have to be feared, it can be economically advantageous to work at relatively high pressure and accordingly at longer residence times compared to atmospheric pressure.

The catalytic oxidation of hydrogen chloride can be carried out adiabatically or preferably isothermally or approximately isothermally, batchwise or preferably continuously as a fixed-bed process at reactor temperatures of from 180 to 500° C., preferably from 200 to 400° C., particularly preferably from 220 to 350° C., and a pressure of from 1 to 25 bar, preferably from 1.2 to 20 bar, particularly preferably from 1.5 to 17 bar and in particular from 2.0 to 15 bar.

In the isothermal or approximately isothermal mode of operation, it is also possible to use a plurality of reactors, i.e. from 2 to 10 reactors, preferably from 2 to 6 reactors, particularly preferably from 2 to 5 reactors, in particular 2 or 3 reactors, connected in series with additional intermediate cooling. The oxygen can either be added together with the hydrogen chloride upstream of the first reactor or its addition can be distributed over the various reactors. This series of arrangements of individual reactors can also be combined in one apparatus.

The process of the invention can in principle be carried out using all known catalysts for the oxidation of hydrogen chloride to chlorine, for example the above-described ruthenium-based catalysts known from DE-A 197 48 299 or DE-A 197 34 412. Further particularly useful catalysts are the gold-based catalysts described in DE-A 102 44 996 which comprise from 0.001 to 30% by weight of gold, from 0 to 3% by weight of one or more alkaline earth metals, from 0 to 3% by weight of one or more alkali metals, from 0 to 10% by weight of one or more rare earth metals and from 0 to 10% by weight of one or more further metals selected from the group consisting of ruthenium, palladium, osmium, iridium, silver, copper and rhenium, on a support, in each case based on the total weight of the catalyst.

A preferred embodiment comprises using a structured catalyst bed in which the catalyst activity increases in the flow direction. Such structuring of the catalyst bed can be achieved by differing impregnation of the catalyst supports with active composition or by differing dilution of the catalyst with an inert material. As inert material, it is possible to use, for example, rings, cylinders or spheres of titanium dioxide, zirconium dioxide or mixtures thereof, aluminum oxide, steatite, ceramic, glass, graphite or stainless steel. In the case of the preferred use of shaped catalyst bodies, the inert material preferably has similar external dimensions.

The region of the gap between the heat-exchange plates nearest the inlet for the gaseous reaction mixture can advantageously be initially charged, in particular to a length of from 5 to 20%, preferably a length of from 5 to 10%, of the total length of the gap with an inert material and only subsequently with the catalyst.

Suitable shaped catalyst bodies can be of any shape; preference is given to pellets, rings, cylinders, stars, wagon wheels or spheres, and particular preference is given to rings, cylinders, star exudates or extruded rods.

Suitable support materials are, for example, silicon dioxide, graphite, titanium dioxide having a rutile or anastase structure, zirconium dioxide, aluminum oxide or mixtures thereof, preferably titanium dioxide, zirconium dioxide, aluminum oxide or mixtures thereof, particularly preferably γ- or δ-aluminum oxide or mixtures thereof.

The supported copper or ruthenium catalyst can, for example, be obtained by impregnation of the support material with aqueous solutions of CuCl₂ or RuCl₃ and, if appropriate, a promoter for doping, preferably in the form of their chlorides. Shaping of the catalyst can be carried out after or preferably before impregnation of the support material.

Suitable promoters for doping are alkali metals such as lithium, sodium, potassium, rubidium and cesium, preferably lithium, sodium and potassium, particularly preferably potassium, alkaline earth metals such as magnesium, calcium, strontium and barium, preferably magnesium and calcium, particularly preferably magnesium, rare earth metals such as scandium, yttrium, lanthanum, cerium, praseodymium and neodymium, preferably scandium, yttrium, lanthanum and cerium, particularly preferably lanthanum and cerium, or mixtures thereof.

The shaped bodies can subsequently be dried and if appropriate calcined at temperatures of from 100 to 400° C., preferably from 100 to 300° C., for example under a nitrogen, argon or air atmosphere. The shaped bodies are preferably firstly dried at from 100 to 150° C. and subsequently calcined at from 200 to 400° C.

The chlorine stream obtained in the process of the invention according to the Deacon process can advantageously be passed to a direct chlorination of ethylene to give 1,2-dichloroethane. This direct chlorination of ethene by means of chlorine is described in DE-A 102 52 859, whose disclosure is hereby fully incorporated by reference into the present patent application.

As an alternative, it is also possible to introduce ethene as additional starting material directly into the reactor in which the gas-phase oxidation of hydrogen chloride by means of the gas stream comprising molecular oxygen is carried out to give 1,2-dichloroethane.

Furthermore, the chlorine stream obtained according to the present invention by the Deacon process can also be passed to a reaction with carbon monoxide to form phosgene, as long as the hydrogen chloride used in the Deacon process has a sufficiently low bromine and iodine content. Such a process is described, for example, in DE-A 102 35 476, whose disclosure is hereby fully incorporated by reference into the present patent application.

As material for the reactor, it is advantageous to choose pure nickel or a nickel-based alloy. As nickel-based alloys, preference is given to using Inconell 600 or Inconell 625. Inconell 600 comprises about 80% of nickel together with about 15% of chromium and also iron. Inconell 625 comprises predominantly nickel, 21% of chromium, 9% of molybdenum and a few percent of niobium. Hastelloy C-276 can also be advantageously used.

All components of the reactor with which the reaction gas mixture comes into contact, in particular distributors, collectors, support grids for the catalyst and also the heat-exchange plates are preferably made of the abovementioned materials pure nickel or nickel-based alloys.

However, it is also possible to manufacture the heat-exchange plates from stainless steel, for example stainless steels having the material numbers 1.4541 or 1.4404, 1.4571 or 1.4406, 1.4539 and also 1.4547 or from other alloy steels.

The temperature profile over the course of the reaction can be discussed in particular detail, in that the process is carried out in a reactor having two or more reaction zones. It is equally possible to carry out the process in two or more separate reactors in place of a single reactor having two or more reaction zones.

In addition or as an alternative, it is also possible to arrange two or more reactors in parallel to one another in the reaction subsection which is prone to hot spots, with the reaction mixture subsequently being combined via a single reactor.

According to the invention, the heat transfer medium used for the indirect removal of the heat of reaction is passed through the heat-exchange plates located in the reactor.

Heat-exchange plates are plate-shaped heat exchangers, i.e. predominantly flat structures which have an interior space which is provided with inlet and outlet lines and has a small thickness relative to its area.

The inlet and outlet facilities for the heat transfer media are generally located at opposite ends of the heat-exchange plates. The heat transfer medium used is frequently water or else Diphyl® (mixture of from 70 to 75% by weight of diphenyl ether and from 25 to 30% by weight of biphenyl), which also partly evaporate in a boiling process; the use of other organic heat transfer media having a low vapor pressure and even ionic liquids is also possible.

The use of ionic liquids as heat transfer media is described in DE-A 103 16 418. Preference is given to ionic liquids containing a sulfate, phosphate, borate or silicate anion. Particularly useful ionic liquids are also ones which contain a monovalent metal cation, in particular an alkali metal cation, and also a further cation, in particular an imidazolium cation. Ionic liquids containing an imidazolium, pyridinium or phosphonium cation are also advantageous.

Plate-shaped heat exchangers are referred to synonymously as heat-exchange plates and also heat transfer plates and heat exchanger plates.

The term heat-exchange plates is used, in particular, for heat transfer plates whose individual, usually two, metal sheets are joined by point and/or rolled seam welding and are frequently plastically molded under hydraulic pressure to obtain a cushion shape.

The term heat-exchange plates will in the present text be used in accordance with the above definition.

In a preferred embodiment, the heat-exchange plates are arranged parallel to one another in the reactor.

In the case of cylindrical reactors, a radial arrangement of the heat-exchange plates to leave a central space and a peripheral channel to the reactor walls free is also advantageous.

The central space which is appropriately contacted with inlet and outlet facilities for the reaction medium to or from the immediate spaces between the heat-exchange plates can in principle have any geometric shape, for example the shape of a rectangle, in particular the shape of a triangle, of a square, of a preferably regular hexagon or of a preferably regular octagon and can also have an essentially circular shape.

The heat-exchange plates preferably extend in the longitudinal direction of the reactor essentially over the entire length of the cylindrical reactor with the exception of the reactor ends.

The reaction medium is preferably conveyed radially through the intermediate spaces between the heat-exchange plates.

The peripheral channel is preferably ring-shaped. It serves as collection and/or distribution chamber for the reaction medium. The peripheral channel can be separated from the intermediate spaces between the heat-exchange plates by a suitable retention device, preferably a cylindrical screen or a perforated plate; analogously, an appropriate retention device can separate the intermediate spaces between the heat-exchange plates from the central space. This embodiment is particularly useful since a reaction is being carried out using a fixed-bed catalyst which is accommodated in the intermediate spaces between the heat-exchange plates and whose discharge with the reaction medium is to be prevented by appropriate choice of the openings in the retention device.

The radial transport of the reaction medium can occur centrifugally or centripetally, with centrifugal transport of the reaction medium being particularly advantageous when the radial flow is in a single direction.

The radial flow of the reaction medium between the radially arranged heat-exchange plates has the advantage of a low pressure drop. Since the oxidation of hydrogen chloride occurs with a decrease in volume, the pressure conditions prevailing in the case of centripetal transport are particularly advantageous because the distances between the heat-exchange plates decrease toward the center.

The radial extension of all heat-exchange plates is preferably identical; fitting of the heat-exchange plates to the interior wall of the reactor is thus not necessary. On the contrary, plates of a single construction type can be used.

The radial extension of the heat-exchange plates is preferably in the range from 0.1 to 0.95 of the reactor radius, particularly preferably in the range from 0.3 to 0.9 of the reactor radius.

The heat-exchange plates are essentially planar. This means that they are not completely flat structures but can be, in particular, regularly curve, folded, creased or corrugated. The heat-exchange plates are produced by known methods.

Periodically profiled structural elements, in particular corrugated plates, may preferably be present in the heat-exchange plates. Such structural elements are known as mixing elements in static mixers and are described, for example, in DE-A 19623051. In the present case, they serve, in particular, to optimize heat transfer.

To match the required heat profile, it is possible to provide a higher plate density in the outer reactor region compared to the inner reactor region, in particular additional plates in the outer reactor region having a smaller radial extension compared to the other heat-exchange plates, preferably with a radial extension in the range from 0.1 to 0.7, particularly preferably from 0.2 to 0.5, of the radial extension of the other heat-exchange plates. The additional plates can each have the same dimensions, but it is also possible to use two or more construction types of additional plates, with the construction types differing from one another in their radial extension and/or their length.

The additional heat-exchange plates are preferably arranged symmetrically between the other heat-exchange plates. They allow improved matching to the temperature profile of the gas-phase oxidation.

A preferred embodiment provides a reactor made up of two or more, in particular detachable, reactor sections. In particular, each reactor section is equipped with a separate heat transfer medium circuit.

The individual reactor sections can be assembled by means of flanges according to requirements. The flow of the reaction medium between two successive reactor sections is preferably achieved by means of suitable deflection plates which have a deflection and/or separation function. Multiple deflection of the reaction medium can be achieved by choosing an appropriate number of deflection plates.

It is possible to provide intermediate introduction points for the reaction medium, in particular via the peripheral channel, on one or more of the reactor sections. In this way, the reaction conditions and the temperature profile can be optimized in an advantageous manner.

It is possible to provide a reactor having a plurality of reactor sections with a single heat transfer medium circuit. However, two or more separate heat transfer medium circuits through the heat-exchange plates in a desired way can also be preferred. In this way, improved matching to different heat transfer requirements as the chemical reaction progresses can be achieved.

The process is preferably carried out in a reactor which is equipped with one or more cuboidal heat-exchange plate modules which are each made up of two or more rectangular heat-exchange plates which are arranged parallel to one another so as to leave a gap between them.

Reactors containing heat-exchange plate modules are known, for example, from DE-A 103 33 866, whose disclosure is hereby fully incorporated by reference into the present patent application.

The heat-exchange plate modules are each made up of two or more rectangular heat-exchange plates which are arranged parallel to one another so as to leave a gap between them.

The material thickness of the metal sheets used for this purpose can be from 1 to 4 mm, from 1.5 to 3 mm, from 2 to 2.5 mm or up to 2.5 mm.

In general, two rectangular metal sheets are joined along their longitudinal sides and ends to form a heat-exchange plate, with a rolled seam or lateral welding shut or a combination of the two being possible, so that the space in which the heat transfer medium is located later is sealed on all sides. The margin of the heat-exchange plates is preferably separated off at or in the lateral rolled seam of the longitudinal edge so that the poorly cooled or uncooled marginal region in which catalyst is usually also present has a very small geometric dimension.

The metal sheets are joined to one another by means of point welds distributed over the rectangular area. At least partial connection by means of straight or curved and even circular rolled seams is also possible. The volume through which the heat transfer medium flows can also be divided into a plurality of separate regions by means of additional rolled seams.

The width of the heat-exchange plates is restricted essentially by manufacturing considerations and can be from 100 to 2500 mm, or from 500 to 1500 mm. The length of the heat-exchange plates depends on the reaction, in particular on the temperature profile of the reaction, and can be from 1000 to 7000 mm, or from 2000 to 6000 mm.

Two or more heat-exchange plates are arranged parallel to one another with a space between them to form a heat-exchange plate module. This results in shaft-like gaps which, at the narrowest points between the plates, have, for example, a width of from 10 to 50 mm, preferably from 15 to 40 mm, more preferably from 18 to 30 mm, in particular 20 mm, between adjacent plates.

The gap can advantageously have a variable width, with narrower gap widths being provided in the regions prone to hot spots compared to the other regions.

Additional spacers can be installed between the individual heat-exchange plates of a heat-exchange plate module, e.g. in the case of large-area plates, to prevent deformations which could alter the spacing or position of the plates. To install these spacers, regions of the plates can be separated off from the flow-through region of the heat transfer medium by means of, for example, circular rolled seams so that, for example, holes for fastening screws of the spacers can be introduced into the plates.

The gaps filled with catalyst particles in a heat-exchange plate module can be sealed from one another, e.g. can be welded shut, or can have a process-side connection to one another.

To set the desired spacing on assembling the individual heat-exchange plates to form a module, the plates are fixed in position so as to fix the distance between them.

The point of welds of adjacent heat-exchange plates can be opposite one another or be offset.

In general, for manufacturing reasons, when two or more cuboidal heat-exchange plate modules are employed, they will each have the same dimensions. In the case of assemblies of 10 or 14 heat-exchange plate modules, it can be advantageous in terms of the compactness of the overall apparatus to choose two module types having different edge lengths or different edge length ratios.

Preference is given to assemblies of 4, 7, 10 or 14 heat-exchange plate modules each having the same dimensions. The visible projection of a module in the flow direction can be square, but can also be rectangular with a side ratio of 1.1 or 1.2. Combinations of 7, 10 or 14 modules having rectangular module projections so that the diameter of the outer cylindrical shell is minimized are advantageous. Particularly advantageous geometric arrangements can be achieved when, as indicated above, a number of 4, 7 or 14 heat-exchange plate modules is chosen.

The heat-exchange plate modules should advantageously be individually replaceable, for example in the case of leaks, deformations of the heat-exchange plates or in the case of problems relating to the catalyst.

The heat-exchange plate modules are advantageously each located in a rectangular stabilizing box.

Each heat-exchange plate module is advantageously held in position by means of a suitable holder, for example by means of the rectangular stabilizing boxes, with a continuous lateral wall or, for example, by means of an angle construction.

In one embodiment, the rectangular stabilizing boxes of adjacent heat-exchange plate modules are sealed from one another. In this way, the reaction mixture cannot flow between the individual heat-exchange plate modules so as to bypass them. The installation of cuboidal heat-exchange plate modules in a predominantly cylindrical reactor leaves relatively large free spaces at the outer edge next to the cylindrical wall. An inert gas can advantageously be fed into this space between the heat-exchange plate modules and the cylindrical wall of the reactor.

The cuboidal heat-exchange plate modules can be installed not only in cylindrical reactors but advantageously also in reactors having polygonal cross sections, in particular rectangular cross sections.

The fixed-bed catalyst is preferably installed in the gaps between the heat-exchange plates in zones having differing catalytic activities in the flow direction of the reaction mixture, preferably with increasing catalytic activity in the flow direction of the reaction gas mixture.

Catalyst particles having equivalent particle diameters in the range from 2 to 8 mm are particularly suitable for the process of the invention. The term equivalent particle diameter refers in a known manner to six times the ratio of volume to surface area of the particle.

The process is particularly advantageously carried out at a superficial velocity of the reaction gas mixture of up to 3.0 m/s, preferably in the range from 0.5 to 2.5 m/s, particularly preferably about 1.5 m/s.

When the reactor in the process of the invention is run up to the reaction temperature and also when the reactor is shut down after the reaction, it is advantageous to pass only an inert flushing gas, preferably nitrogen, which has been heated to a temperature above the condensation point of hydrochloric acid through the reactor at temperatures in the reactor below 150° C. For the present purposes, gases are considered to be inert if they do not react with the substances intrinsic to the process under the operating conditions of the process of the invention. This particular procedure during the start-up and shutdown of the reactor avoids corrosion damage to the material of construction of the reactor.

The invention is illustrated below with the aid of a drawing.

In the individual figures:

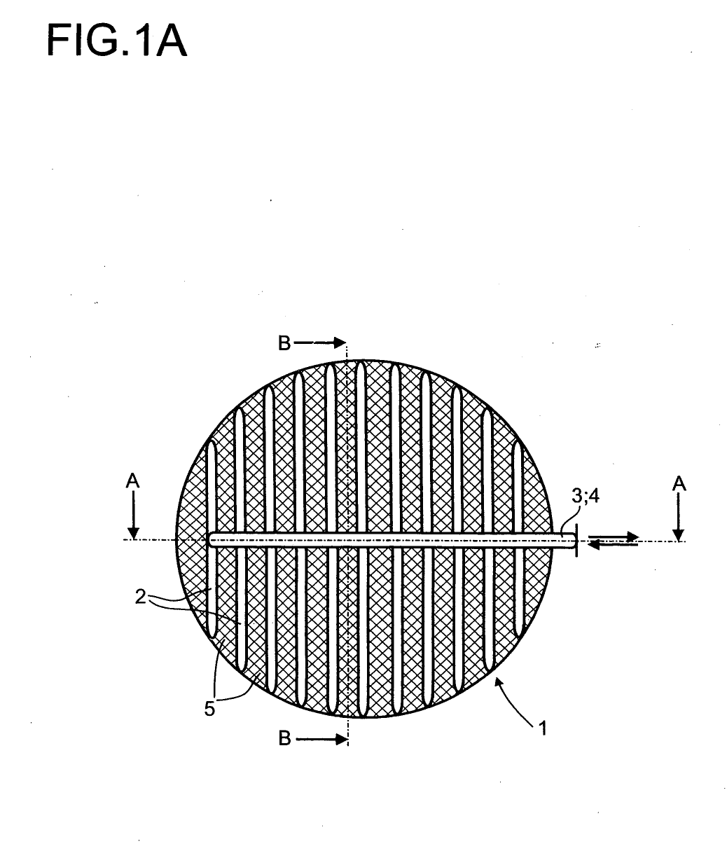

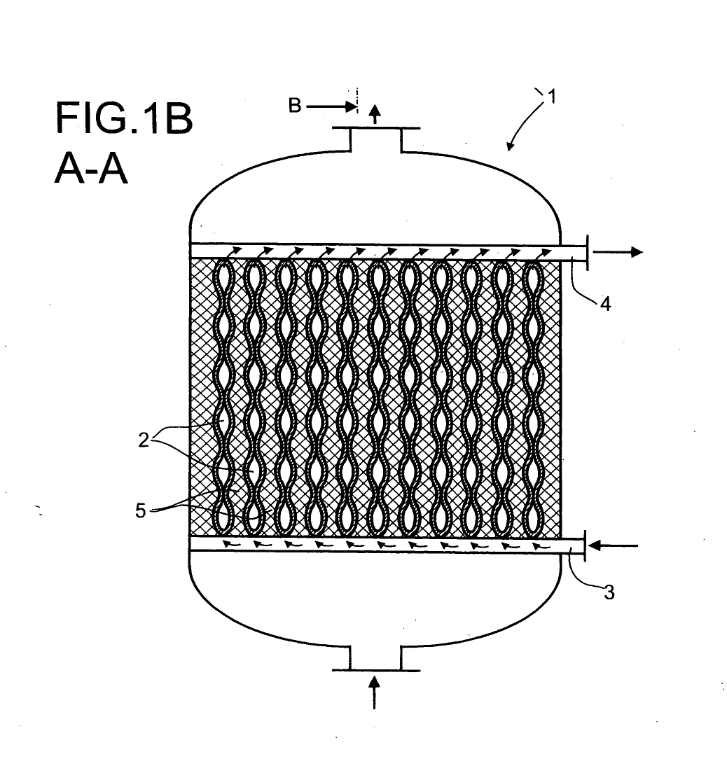

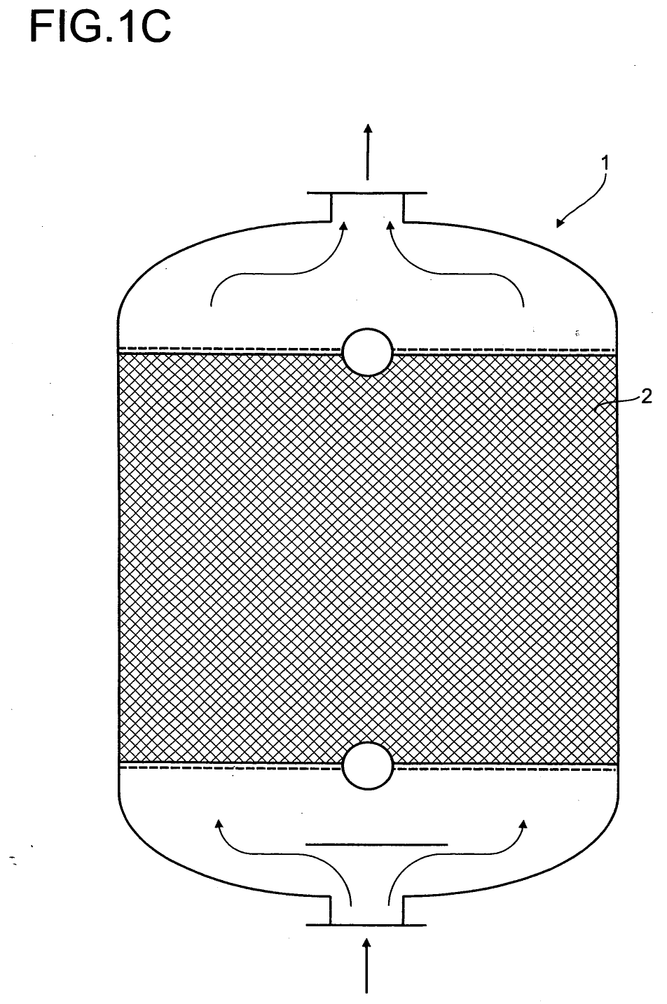

FIG. 1A shows a preferred embodiment of a reactor for the process of the invention, cross section, with longitudinal section shown in FIG. 1B and longitudinal section through a heat-exchange plate in FIG. 1C,

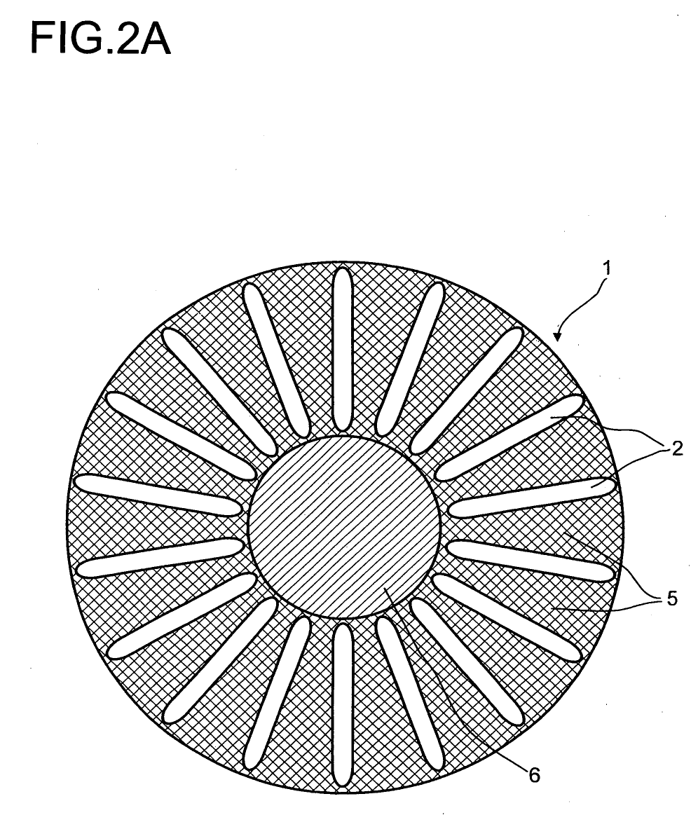

FIG. 2A shows a cross section through a further, preferred embodiment of a reactor for the process of the invention, with longitudinal sections shown in FIG. 2B and a variant with a plurality of reaction sections in FIG. 2C,

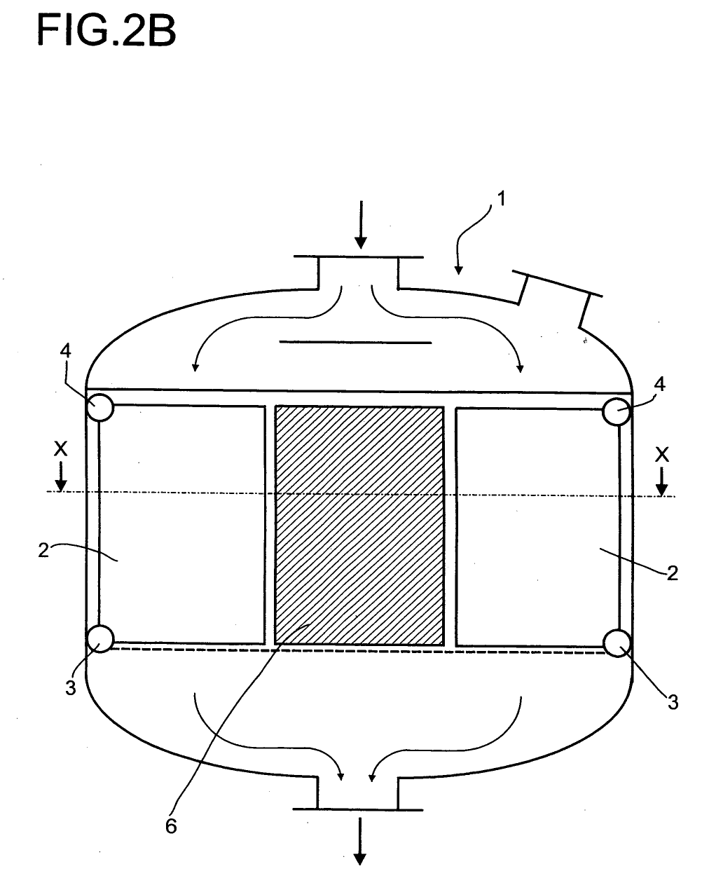

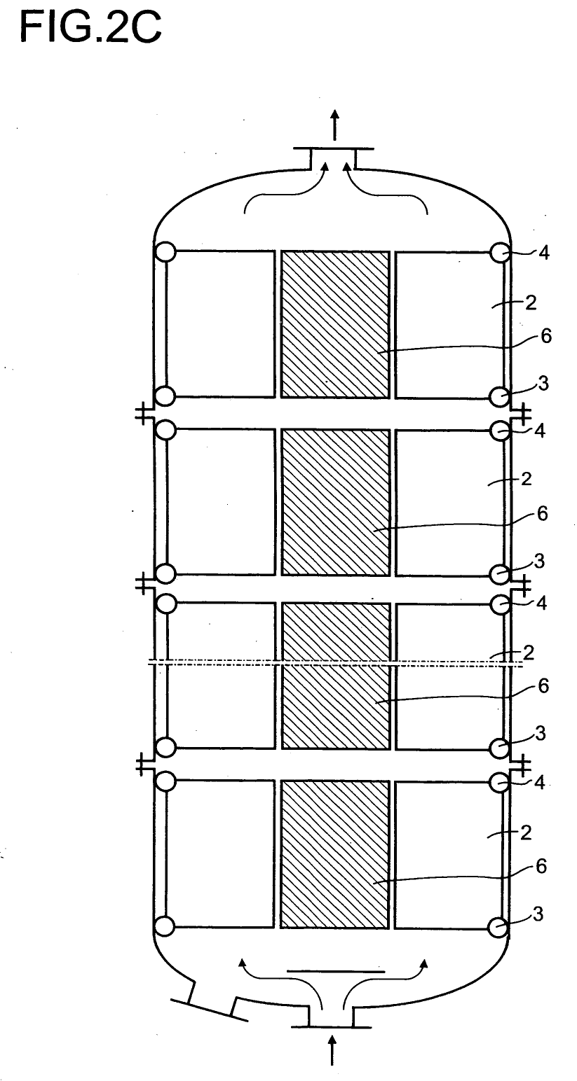

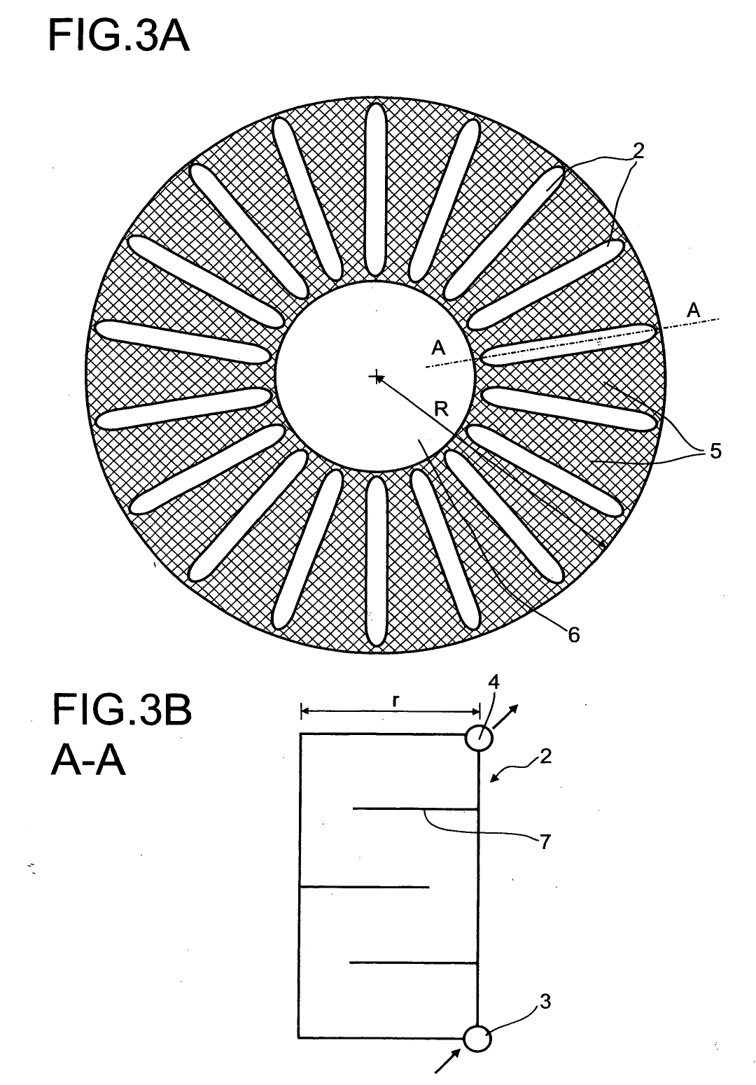

FIG. 3A shows a further, preferred embodiment in cross section, with longitudinal section through a heat-exchange plate shown in FIG. 3B,

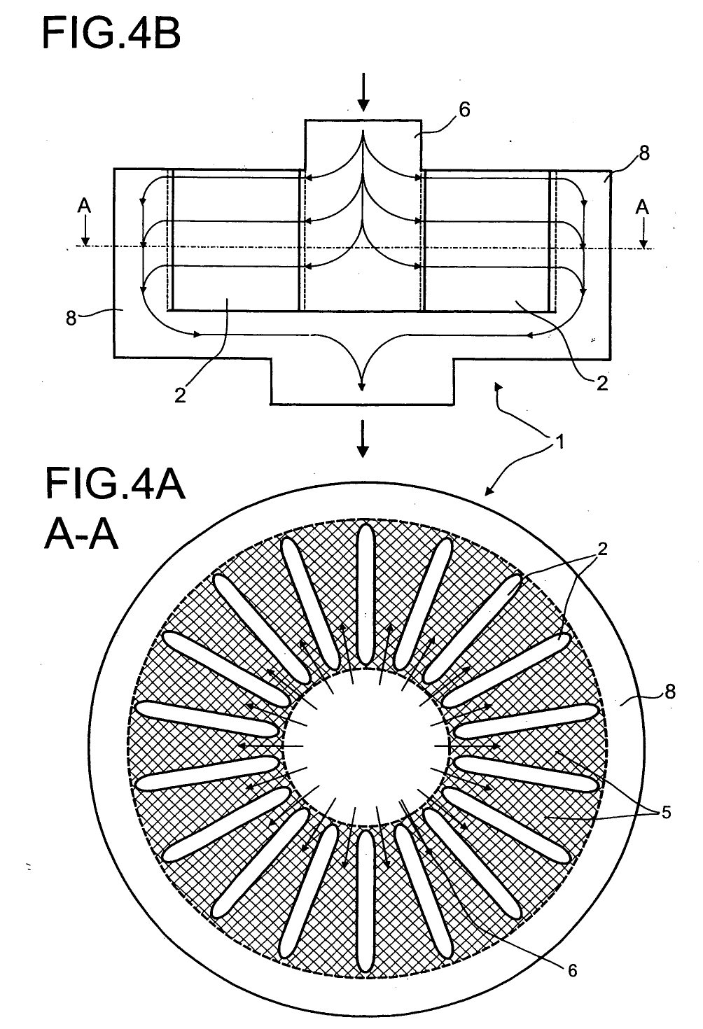

FIG. 4A shows another embodiment of a reactor for the process of the invention, with longitudinal sections shown in FIG. 4B and a variant with a plurality of reaction sections in FIG. 4C,

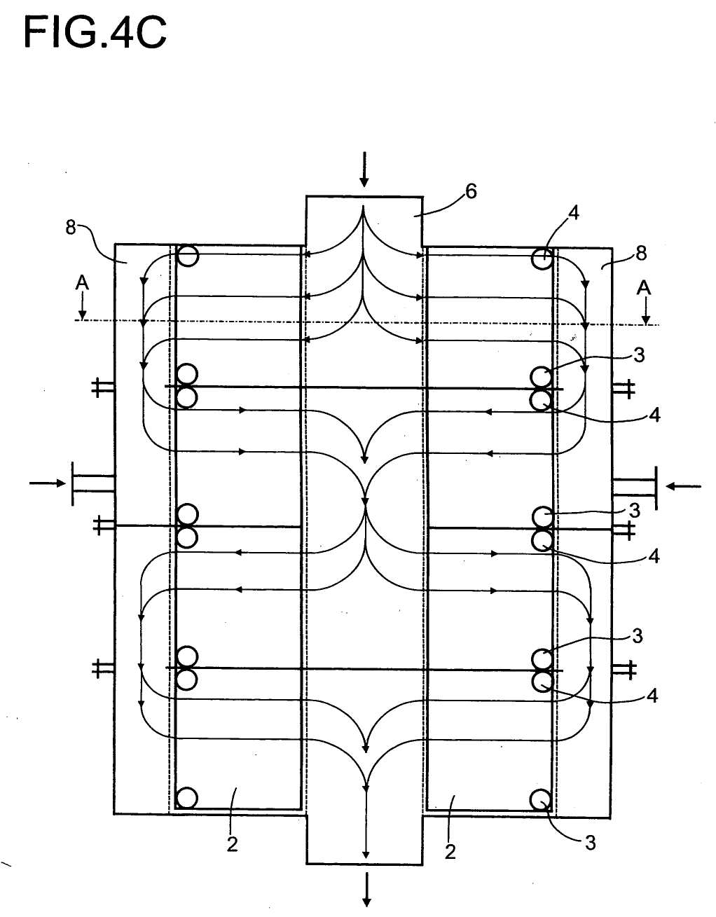

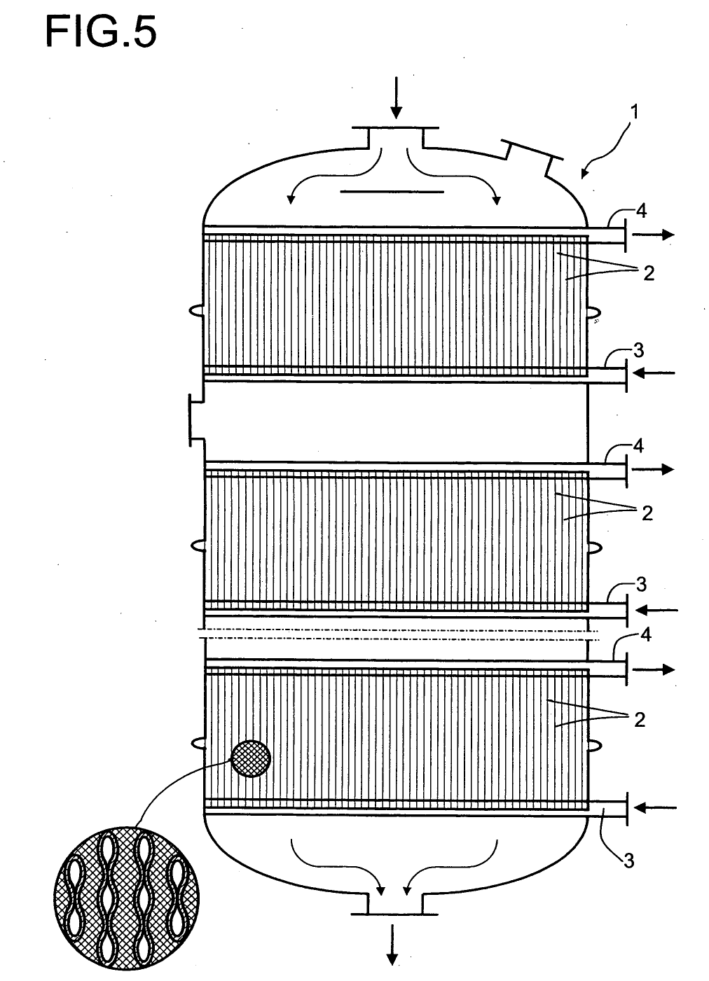

FIG. 5 shows an embodiment of a reactor for the process of the invention, in longitudinal section,

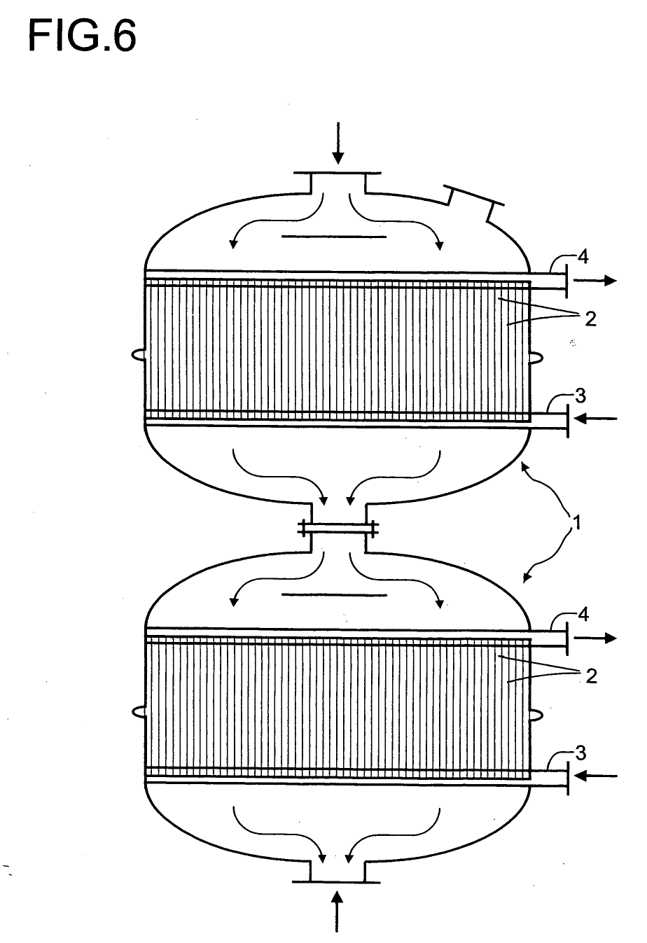

FIG. 6 shows an further embodiment for two reactors connected in series,

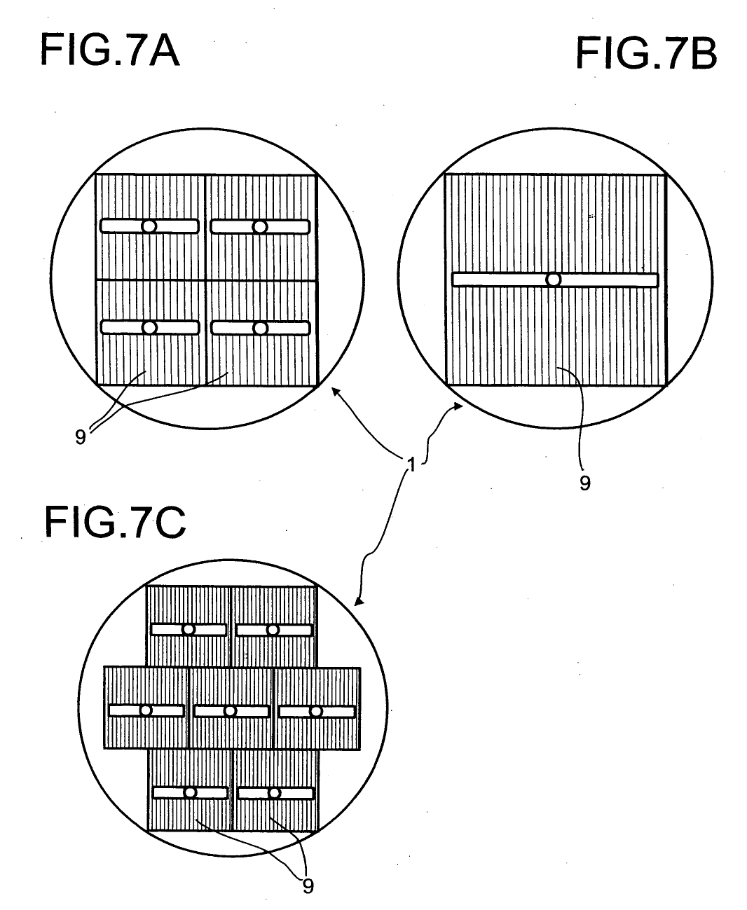

FIGS. 7A to 7C show different arrangements of heat-exchange plate modules, in cross section, and

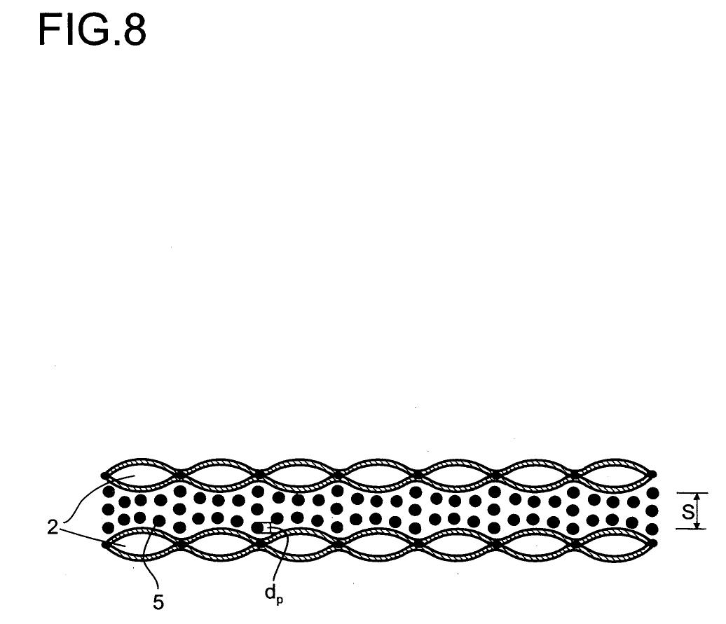

FIG. 8 shows a gap between heat-exchange plate modules.

The cross-sectional depiction in FIG. 1A shows a section through a reactor 1 having parallel heat-exchange plates 2 which are arranged therein and leave the gap 5 free between the heat-exchange plates, with the gap 5 being charged with a solid catalyst. Inlet and outlet lines 3 and 4, respectively, are provided for the heat transfer medium circulating through the heat-exchange plates 2.

The longitudinal section shown in FIG. 1B illustrates the configuration of the heat-exchange plates 2 and the arrangement of inlet and outlet lines 3 and 4, respectively, in the reactor 1. A mode of operation with the reaction gas being passed from the bottom upward is shown by way of example; the reverse direction of flow from the top downward is equally possible.

FIG. 1C shows a longitudinal section through a heat-exchange plate 2. Retention devices for the solid catalyst at the two ends of the heat-exchange plate 2 are also shown in the figure.

The section depicted in FIG. 2A shows a reactor 1 with heat-exchange plates 2 arranged radially therein and gaps 5 which are charged with the solid catalyst between the heat-exchange plates 2.

A dummy body is located in the central space 6 to ensure essentially longitudinal flow of the reaction mixture through the reactor as indicated, in particular, by the arrows in the longitudinal section shown in FIG. 2B.

The longitudinal section depicted in FIG. 2C shows a variant of the apparatus shown in longitudinal section in FIG. 2B, with a plurality of, for example, four, reactor sections.

FIG. 3A shows a cross section through a further embodiment of a reactor for the process of the invention, without a dummy body in the central space 6. R denotes the radius of the reactor and r denotes the extension of each heat-exchange plate in the direction of the reactor radius R. The cross section through a heat-exchange plate 2 depicted in FIG. 3B shows deflection plate 7 for the heat transfer medium.

The cross section depicted in FIG. 4A shows a further embodiment having a peripheral channel 8 for collecting the reaction gas mixture and passing it on. The longitudinal section depicted in FIG. 4B illustrates the flow profile for the reaction gas mixture, in particular through the central space 6 and the peripheral channel 8.

The longitudinal section depicted in FIG. 4C shows a further variant having a plurality of, for example, two, reactor sections arranged in series.

The longitudinal section depicted in FIG. 5 shows a reactor 1 with, by way of example, three reactor sections each provided with heat-exchange plates 2 and inlet and outlet lines 3 and 4, respectively, for the heat transfer medium.

The longitudinal section depicted in FIG. 6 shows two reactors 1 connected in series, each provided with heat-exchange plates 2 and inlet and outlet lines 3 and 4, respectively, for the heat transfer medium.

FIGS. 7A to 7C show assemblies of 4, 1 and 7 heat-exchange plate modules 9 in a cylindrical reactor 1, in cross section.

FIG. 8 illustrates the configuration of the heat-exchange plates 2 and the gap 5 located between them, with fixed-bed catalyst having an equivalent particle diameter dₚ present therein. It can be seen from the figure that the width s of the gap 5 is the smallest distance between two adjacent heat-exchange plates 2.

1: A process for preparing chlorine by gas-phase oxidation of hydrogen chloride by means of a gas stream comprising molecular oxygen in the presence of a fixed-bed catalyst, wherein the process is carried out in a reactor having heat-exchange plates that are arranged in the longitudinal direction of the reactor and have a spacing between them and through which a heat transfer medium flows, inlet and outlet facilities for the heat transfer medium to the heat-exchange plates and also gaps between heat-exchange plates in which the fixed-bed catalyst is present and into which the hydrogen chloride and the gas stream comprising molecular oxygen are passed.

2: A process according to claim 1, wherein the product gas stream taken from the reactor is passed to a direct chlorination of ethylene to form 1,2-dichloroethane.

3: A process according to claim 1, wherein ethylene is fed as further starting material into the reactor, with 1,2-dichloroethane being obtained as desired product in the reactor.

4: A process according to claim 1, wherein the heat-exchange plates are arranged parallel to one another in the reactor.

5: A process according to claim 1, wherein the reactor is cylindrical and the heat-exchange plates are arranged radially to leave a central space and a peripheral channel free in the cylindrical reactor and the gas stream comprising hydrogen chloride.

6: A process according to claim 5, wherein the radial extension of the heat-exchange plates is from 0.1 to 0.95 of the reactor radius.

7: A process according to claim 1, wherein the reactor is made up of two or more reactor sections.

8: A process according to claim 1, wherein the reactor is equipped with one or more cuboidal heat-exchange plate modules that are each made up of two or more rectangular heat-exchange plates that are arranged parallel to one another so as to leave a gap between them.

9: A process according to claim 8, wherein the reactor has two or more cuboidal heat-exchange plate modules, each having identical dimensions.

10: A process according to claim 9, wherein the reactor has 4, 7, 10 or 14 heat-exchange plate modules.

11: A process according to claim 1, wherein the heat-exchange plates are each made up of two rectangular metal sheets that are joined on their longitudinal sides and ends by rolled seam welding and the margin of the metal sheets projecting beyond the rolled seam is separated off at the outer edge of the rolled seam or in the rolled seam itself.

12: A process according to claim 8, wherein the reactor is cylindrical and an inert gas is fed into the space between the heat-exchange plate modules and the cylindrical wall of the reactor.

13: A process according to claim 1, wherein the fixed-bed catalyst in the gaps is arranged in zones having a differing catalytic activity.

14: A process according to claim 1, wherein a fixed-bed catalyst made up of particles with an equivalent particle diameter in the range from 2 to 8 mm is used.

15: A process according to claim 1, wherein the width of the gap is in the range from 10 to 50 mm, and the ratio of the width of the gap to the equivalent particle diameters is from 2 to 10.

16: A process according to claim 1, wherein the superficial velocity of the reaction gas mixture in the gaps is up to 3.0 m/s.

17: A process according to claim 1, wherein the reaction gas mixture and the heat transfer medium are conveyed in cocurrent through the reactor.

18: A process according to claim 1, wherein only a preheated inert flushing gas, in particular nitrogen, is passed through the reactor at temperatures below 150° C. during the start-up and shutdown of the reactor.

19: A process according to claim 5, wherein molecular oxygen is fed radially into the gap between the heat exchange plates.

20: A process according to claim 6, wherein the radial extension of the heat-exchange plates is from 0.3 to 0.9 of the reactor radius.
